(19) United States
(12) Patent Application Publication
(10) Pub. No.: US 2017/0017462 A1
(43) Pub. Date: Jan. 19, 2017

(54) AUDIO SYSTEM AND METHOD FOR ADAPTIVE SOUND PLAYBACK DURING PHYSICAL ACTIVITIES

(71) Applicant: Fraunhofer-Gesellschaft zur Foerderung der angewandten Forschung e.V., Muenchen (DE)

(72) Inventors: Aleksandra Maria KUBIAK, Oldenburg (DE); Jan RENNIES-HOCHMUTH, Oldenburg (DE); Jens-Ekkehart APPELL, Achternmeer (DE)

(21) Appl. No.: 15/282,428

(22) Filed: Sep. 30, 2016

Related U.S. Application Data
(63) Continuation of application No. PCT/EP2014/057326, filed on Apr. 10, 2014.

(51) Int. Cl. G06F 3/16; A61B 5/0205; H04R 29/00; H03G 3/30; H04R 3/04

(52) U.S. Cl. CPC G06F 3/165; H03G 3/3005; H04R 3/04; H04R 29/001; A61B 5/02055; A61B 5/021

(57) ABSTRACT

The invention provides an audio system for adaptive audio playback during physical activities including:

an audio player having one or more audio sources and being configured to output an audio signal based on one or more source signals provided by the one or more audio sources; and
a control device configured to control one or more settings of the of the audio player, which affect the audio signal;
wherein the control device includes a user interface configured to input user preferences for the one or more settings of the audio player;
wherein the control device includes a processor configured to correlate user preferences and values of one or more sensor signals corresponding to a physical strain of a user of the audio system, further configured to store the user preferences and the correlated values of the one or more sensor signals in a memory of the control device and further configured to control the one or more settings of the audio player based on the one or more sensor signals and based on the user preferences and the correlated values of the one or more sensor signals stored in the memory.

### CROSS-REFERENCE TO RELATED APPLICATIONS

This application is a continuation of copending International Application No. PCT/EP2014/057326, filed Apr. 10, 2014, which is incorporated herein by reference in its entirety.

### BACKGROUND OF THE INVENTION

Millions of people exercise to music every day, which can involve both listening via headphones as well as listening via loudspeakers. Many active sportsmen/sportswomen report that their perception of sound changes with increasing degree of physical exercise. This can involve an increased sensitivity to loud sounds or a particularly perceived annoyance of, e.g., high-frequency sounds. Reported countermeasures include: turning the volume down, changing types of music, changing equalizer settings and/or switching music off.

In any case, each of these countermeasures necessitates an explicit action of the individual user when using a conventional audio system.

Furthermore, the individual vulnerability to an increased discomfort and the countermeasure can differ widely between users.

### SUMMARY

According to an embodiment, an audio system for adaptive audio playback during physical activities may have: an audio player having one or more audio sources and being configured to output an audio signal based on one or more source signals provided by the one or more audio sources; and a control device configured to control one or more settings of the of the audio player, which affect the audio signal; wherein the control device includes a user interface configured to input user preferences for the one or more settings of the audio player; wherein the control device includes a processor configured to correlate user preferences and values of one or more sensor signals corresponding to a physical strain of a user of the audio system, further configured to store the user preferences and the correlated values of the one or more sensor signals in a memory of the control device and further configured to control the one or more settings of the audio player based on the one or more sensor signals and based on the user preferences and the correlated is values of the one or more sensor signals stored in the memory.

According to another embodiment, a method for adaptive audio playback during physical activities may have the steps of: providing an audio player having one or more audio sources and being configured to output an audio signal based on one or more source signals provided by the one or more audio sources; and providing a control device configured to control one or more settings of the of the audio player, which affect the audio signal; providing one or more sensor signals corresponding to a physical strain of a user of the audio system by using one or more sensors of the control device; inputting user preferences for the one or more settings of the audio player at a user interface of the control device; correlating user preferences and values of the one or more sensor signals by using a processor of the control device; storing the user preferences and the correlated values of the one or more sensor signals in a memory of the control device by using the processor of the control device; and controlling the one or more settings of the audio player based on the one or more sensor signals and based on the user preferences and the correlated values of the one or more sensor signals stored in the memory by using the processor of the control device.

Another embodiment may have a non-transitory digital storage medium having a computer program stored thereon to perform the method for adaptive audio playback during physical activities, the method having the steps of: providing an audio player having one or more audio sources and being configured to output an audio signal based on one or more source signals provided by the one or more audio sources; and providing a control device configured to control one or more settings of the of the audio player, which affect the audio signal; providing one or more sensor signals corresponding to a physical strain of a user of the audio system by using one or more sensors of the control device; inputting user preferences for the one or more settings of the audio player at a user interface of the control device; correlating user is preferences and values of the one or more sensor signals by using a processor of the control device; storing the user preferences and the correlated values of the one or more sensor signals in a memory of the control device by using the processor of the control device; and controlling the one or more settings of the audio player based on the one or more sensor signals and based on the user preferences and the correlated values of the one or more sensor signals stored in the memory by using the processor of the control device, when said computer program is run by a computer.

The audio player may be of any known types of analogous or digital audio players. The audio signal provided by the audio player may be fed directly or indirectly to headphones, loudspeakers or other devices in order to create an audible sound.

The control device may be any analogous or digital control device capable of controlling at least one setting of the audio player, which has an effect to the output audio signal. The term “setting” refers herein to any variable parameter, which is used by the audio player in order to create the output signal.

Furthermore, the at least one sensor signal may be any sensor signal which directly or indirectly corresponds to a physical restraint or physical stress of the user of the audio system. The user interface may be of any known type, for example a keyboard or touch screen.

According to the invention the control device comprises a processor, to which the at least one sensor signal is fed and to which the user interface is connected to. The term “processor” refers to a hardware, a software or a combination of hardware and software, which is capable of executing the following tasks: correlating user preferences and values of the one or more sensor signals storing the user preferences and the correlated values of the one or is more sensor signals in a memory of the control device and controlling the one or more settings of the audio player based on the one or more sensor signals and based on the user preferences and the correlated values of the one or more sensor signals stored in the memory.

By correlating user preferences and values of the at least one sensor signal the processor collects information about the individual perception of sounds under physical strain of the user. As the user preferences and the correlated values of the at least one sensor signal are stored, the information about individual perception of sound may later be used in order to adapt the audio signal output by the audio player. The more often the user inputs user preferences in different physical activity conditions the collected information gets more and more accurate.

By controlling the one or more settings of the audio player based on the one or more sensor signals and based on the user preferences and the correlated values of the one or more sensor signals stored in the memory, the audio signal may be adapted to the physical strain condition of the user, which is represented by the sensor signals, taking into account the individual perception of sounds of the user under physical strain, which is represented by the user preferences and the correlated values of the sensor signals stored in the memory.

The audio system according to the invention may significantly improve the comfort of any user by reducing the need to re-adjust the settings of the audio player when the intensity of a physical stress changes. Moreover, the comfort of any user may be improved by automatically avoiding audio player settings which may result in uncomfortable sounds during physical activities.

According to an embodiment of the invention the one or more sensor signals comprise one or more sensor signals of one or more bio sensors configured to detect a biological parameter of the user.

According to an embodiment of the invention the one or more signals of the one or more bio sensors comprise a signal of a body temperature sensor, a signal of a pulse sensor, a signal of a respiratory movement sensor, a signal of a respiratory flow sensor, a signal of a pulse oximetry sensor, a signal of a blood pressure meter, a signal of a capnograph and/or a signal of a maximal oxygen uptake sensor.

According to an embodiment of the invention the one or more sensor signals comprise one or more signals of one or more activity sensors configured to detect a parameter related to a physical activity of the user.

According to an embodiment of the invention the one or more signals of one or more activity sensors comprise a signal of a pedometer, a signal of a distance meter and/or a signal of an activity time keeper.

According to an embodiment of the invention the control device comprises one or more sensors configured to provide the one or more sensor signals corresponding to a physical strain of a user of the audio system.

According to an embodiment of the invention the one or more sensors comprise one or more bio sensors configured to detect a biological parameter of the user. The term “bio sensor” as used herein refers to a device for measuring a physical and chemical life processes at or within human beings. Such sensors allow detecting a physical strain of the user of the audio system directly and in real time.

According to an embodiment of the invention the one or more bio sensors comprise a body temperature sensor, a pulse sensor, a respiratory movement sensor, a respiratory flow sensor, a pulse oximetry sensor, a blood pressure meter, a capnograph and/or a maximal oxygen uptake sensor.

According to an embodiment of the invention the one or more sensors comprise one or more activity sensors configured to detect a parameter related to a physical activity of the user. Such parameters include all parameters that describe the duration, intensity and/or type of the activity. Activity sensors allow detecting the physical strain of the user of the audio system in direct as the eventual activity results in a physical strain. Activity sensors and bio-sensors both may be used exclusively or in combination with each other.

According to an embodiment of the invention the one or more activity sensors comprise a pedometer, a distance meter and/or an activity time keeper.

According to an embodiment of the invention the one or more settings comprise a volume control setting, an equalizer control setting, an audio source selecting setting, a dynamic range compression control setting and/or an audio content selecting setting.

The volume control setting may be used to set the volume of the audio signal at a volume control of the audio player. By adapting the volume control setting, the volume may be controlled automatically according to the individual perception of the volume of the user under physical stress.

The equalizer control setting may be used to control the frequency response of the audio player at an equalizer of the audio player. By adapting the equalizer control setting, the frequency distribution of the audio signal may be controlled automatically according to the individual perception of a frequency distribution of the user under physical stress.

The dynamic range compression control setting may be used to set the dynamic range of the audio signal at a compressor/expander of the audio player. This will affect, amongst others, perceived loudness and clarity. By adapting the dynamic range compression control setting, the compression/expansion may be controlled automatically according to the individual perception of the user under physical stress.

An audio source selecting setting may be used to select a source signal for further processing in such embodiments, wherein the audio player comprises a plurality of audio sources, at a source selecting stage of the audio player.

The select signal may be forwarded to the volume control, then forwarded as a volume controlled signal to the equalizer and then forwarded to the compressor/expander as a volume controlled and equalized signal. However the order of the volume control, the equalizer and/or the compressor/expander may be changed.

Furthermore, an audio content selecting setting may be used for controlling the content of the source signal of a certain audio source. The term “audio content selecting” refers inter alia to selecting a type of content, e.g. speech or music, to selecting a genre of speech, e.g. news or a fiction, to selecting a genre of music, e.g. beat or soul, to selecting music having specific parameters, e.g. rhythm and speed, or to selecting of a predefined playlist. By adapting the audio content selecting setting, the audio content of the audio signal may be controlled automatically according to the individual perception of audio content of the user under physical stress

According to an embodiment of the invention the one or more audio sources comprise an audio data receiver configured for providing the source signal based on remote audio data. Remote audio data are such audio data not stored locally at the audio player. The receiver may be an analogous or a digital radio receiver including internet radio receivers or a receiver configured to receive other internet services.

According to an embodiment of the invention the one or more audio sources comprise an audio data reader configured for providing the source signal based on local audio data. The audio data reader for example may be an MP3 decoder or a wav file decoder. The audio data may be retrieved from a locally available audio data carrier such as from a hard drive, a solid-state memory, a CD or a DVD.

According to an embodiment of the invention the processor is configured to control the one or more settings of the audio player based on one or more measuring signals corresponding to physical characteristics of ambient sound.

According to an embodiment of the invention the control device comprises one or more sensors configured for providing the one or more measuring signals corresponding to the physical characteristics of ambient sound, wherein the processor is configured to control the one or more settings of the audio player based on the physical characteristics of ambient sound. By these features the audio signal may be adapted to the amount and/or characteristics of environmental sound.

According to an embodiment of the invention the processor is configured to correlate user preferences and values of the physical characteristics of ambient sound, further configured to store the user preferences and the correlated values of the physical characteristics of ambient sound in the memory and further configured to control the one or more settings of the audio player based on the user preferences and the correlated values of the physical characteristics of ambient sound stored in the memory. By these features the audio signal may be adapted according to the individual perception of ambient sounds by the user.

The object of the invention is further achieved by a method for adaptive audio playback during physical activities comprising the steps of:

providing an audio player having one or more audio sources and being configured to output an audio signal based on one or more source signals provided by the one or more audio sources; and
providing a control device configured to control one or more settings of the audio player, which affect the audio signal;
providing one or more sensor signals corresponding to a physical strain of a user of the audio system;
inputting user preferences for the one or more settings of the audio player at a user interface of the control device;
correlating user preferences and values of the one or more sensor signals by using a processor of the control device;
storing the user preferences and the correlated values of the one or more sensor signals in a memory of the control device by using the processor of the control device; and
controlling the one or more settings of the audio player based on the one or more sensor signals and based on the user preferences and the correlated values of the one or more sensor signals stored in the memory by using the processor of the control device.

Moreover, the object of the invention is achieved by a computer program for performing, when running on a computer or a processor, the inventive method.

### BRIEF DESCRIPTION OF THE DRAWINGS

Embodiments of the present invention will be detailed subsequently referring to the appended drawings, in which:

FIG. 1 illustrates a first embodiment of an audio system according to the invention in a schematic view;

FIG. 2 illustrates a second embodiment of an audio system according to the invention in a schematic view; and

FIG. 3 illustrates an embodiment of a control device of an audio system according to the invention in a schematic view.

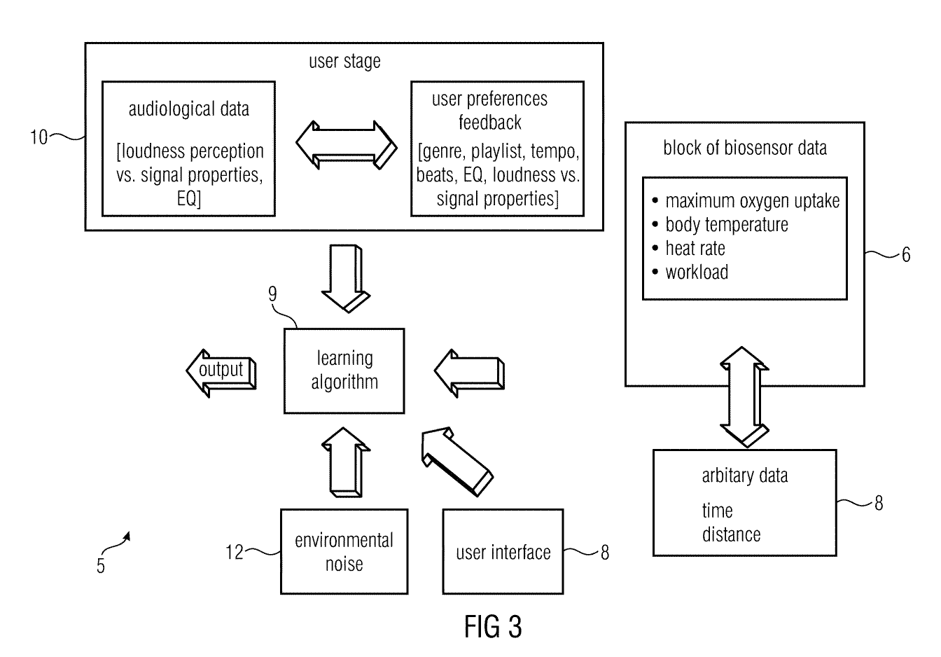

### DETAILED DESCRIPTION OF THE INVENTION

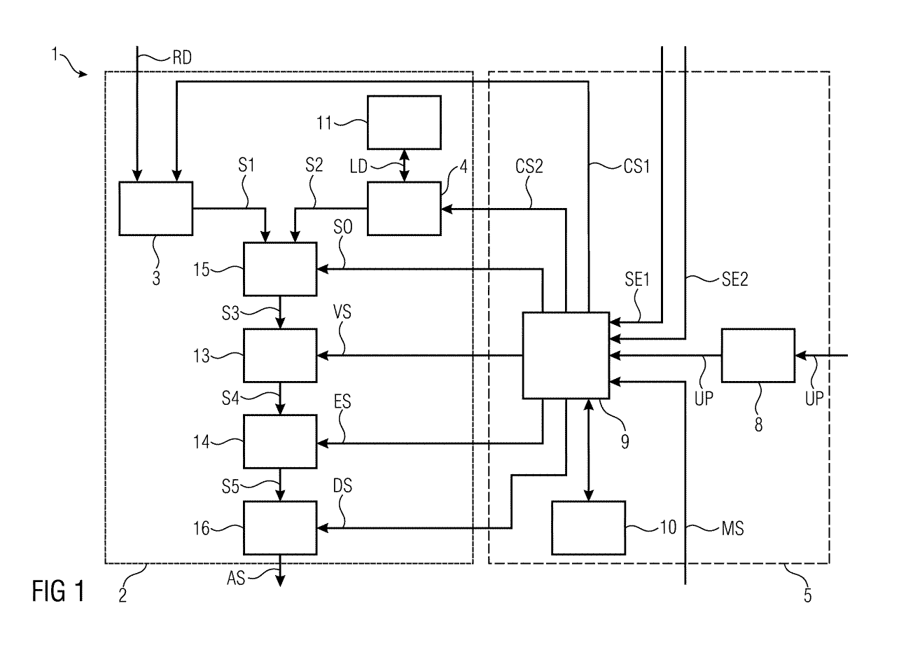

FIG. 1 illustrates a first embodiment of an audio system 1 for adaptive audio playback during physical activities comprising:

an audio player 2 having one or more audio sources 3, 4 and being configured to output an audio signal AS based on one or more source signals S1, S2 provided by the one or more audio sources 3, 4; and
a control device 5 configured to control one or more settings VS, ES, SO, CS1, CS2 of the of the audio player 2, which affect the audio signal AS;
wherein the control device 5 comprises a user interface 8 configured to input user preferences UP for the one or more settings VS, ES, SO, CS1, CS2 of the audio player 2;
wherein the control device 5 comprises a processor 9 configured to correlate user preferences UP and values of one or more sensor signals SE1, SE2 corresponding to a physical strain of the user of the audio system 1, further configured to store the user preferences UP and the correlated values of the one or more sensor signals SE1, SE2 in a memory 10 of the control device 5 and further configured to control the one or more settings VS, ES, SO, CS1, CS2 of the audio player based on the one or more sensor signals SE1, SE2 and based on the user preferences UP and the correlated values of the one or more sensor signals SE1, SE2 stored in the memory 10.

The audio player 2 may be of any known types of analogous or digital audio players 2. The audio signal AS provided by the audio player 2 may be fed directly or indirectly to headphones, loudspeakers or other devices in order to create an audible sound.

The control device 5 may be any analogous or digital control device 5 capable of controlling at least one setting VS, ES, SO, CS1, CS2 of the audio player 2, which has an effect to the output audio signal AS. The term “setting” refers herein to any variable parameter, which is used by the audio player 2 in order to create the output signal AS.

Furthermore, the at least one sensor signal SE1, SE2 may be any sensor signal SE1, SE2, which directly or indirectly corresponds to a physical restraint or physical stress of the user of the audio system 1. The user interface 8 may be of any known type, for example a keyboard or touch screen.

According to the invention the control device 5 comprises a processor 9, to which the at least one sensor signal SE1, SE2 is fed and to which the user interface 8 is connected to. The term “processor” refers to a hardware, a software or a combination of hardware and software, which is capable of executing the following tasks: correlating user preferences UP and values of the one or more sensor signals SE1, SE2, storing the user preferences UP and the correlated values of the one or more sensor signals SE1, SE2 in a memory 10 of the control device 5 and controlling the one or more settings VS, ES, SO, CS1, CS2 of the audio player based on the one or more sensor signals SE1, SE2 and based on the user preferences UP and the correlated values of the one or more sensor signals SE1, SE2 stored in the memory time.

By correlating user preferences UP and values of the at least one sensor signal SE1, SE2 the processor 9 collects information about the individual perception of sounds of the user under physical strain. As the user preferences UP and the correlated values of the at least one sensor signal SE1, SE2 are stored, the information about individual perception of sound may later be used in order to adapt the audio signal AS output by the audio player 2. The more often the user inputs user preferences UP in different physical activity conditions the collected information gets more and more accurate.

By controlling the one or more settings VS, ES, SO, CS1, CS2 of the audio player 2 based on the one or more sensor signals SE1, SE2 and based on the user preferences UP and the correlated values of the one or more sensor signals SE1, SE2 stored in the memory 10, the audio signal AS may be adapted to the physical strain condition of the user, which is represented by the sensor signals SE1, SE2, taking into account the individual perception of sounds of the user under physical strain, which is represented by the user preferences UP and the correlated values of the sensor signals SE1, SE2 stored in the memory 10.

The audio system 1 according to the invention may significantly improve the comfort of any user by reducing the need to re-adjust the settings VS, ES, SO, CS1, CS2 of the audio player 2 when the intensity of a physical stress changes. Moreover, the comfort of any user may be improved by automatically avoiding audio player settings VS, ES, SO, CS1, CS2 which may result in uncomfortable sounds during physical activities.

According to an embodiment of the invention the one or more sensor signals SE1, SE2 comprise one or more sensor signals SE1 of one or more bio sensors configured to detect a biological parameter of the user.

According to an embodiment of the invention the one or more signals SE1 of the one or more bio sensors comprise a signal of a body temperature sensor, a signal of a pulse sensor, a signal of a respiratory movement sensor, a signal of a respiratory flow sensor, a signal of a pulse oximetry sensor, a signal of a blood pressure meter, a signal of a capnograph and/or a signal of a maximal oxygen uptake sensor.

According to an embodiment of the invention the one or more sensor signals SE1, SE2 comprise one or more signals SE2 of one or more activity sensors configured to detect a parameter related to a physical activity of the user.

According to an embodiment of the invention the one or more signals SE2 of one or more activity sensors comprise a signal of a pedometer, a signal of a distance meter and/or a signal of an activity time keeper.

According to an embodiment of the invention the processor 9 is configured to control the one or more settings of the audio player based on one or more measuring signals MS corresponding to physical characteristics of ambient sound.

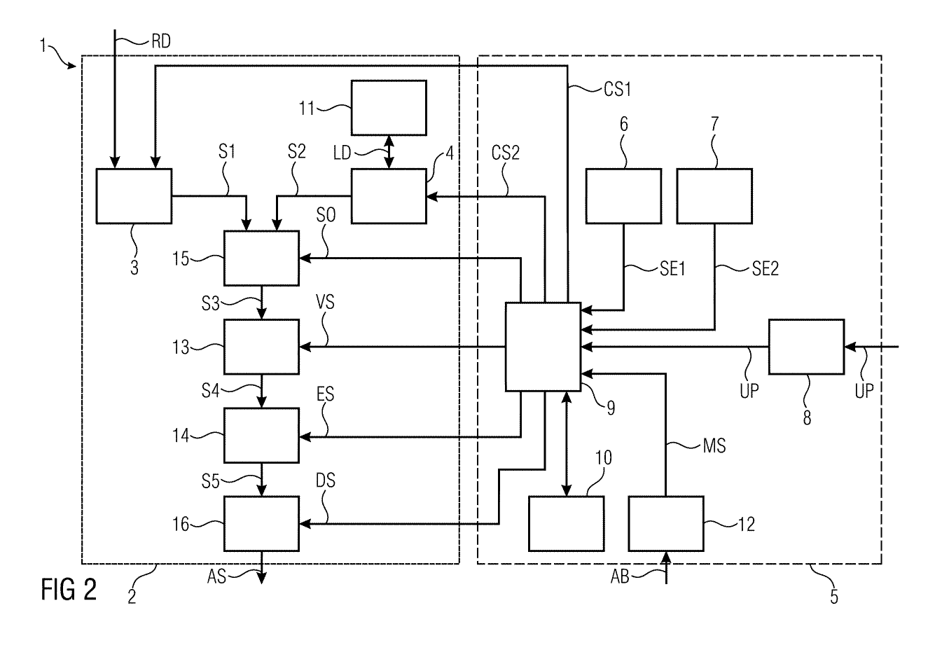

FIG. 2 illustrates a second embodiment of an audio system 1 for adaptive audio playback during physical activities.

According to an embodiment of the invention the control device 5 comprises one or more sensors 6, 7 configured to provide the one or more sensor signals SE1, SE2 corresponding to a physical strain of a user of the audio system 1.

According to an embodiment of the invention the one or more sensors 6, 7 comprise one or more bio sensors 6 configured to detect a biological parameter of the user. The term “bio sensor” as used herein refers to a device for measuring a physical and chemical life processes at or within human beings. Such sensors 6 allow detecting a physical strain of the user of the audio system 1 directly and in real time.

According to an embodiment of the invention the one or more bio sensors 6 comprise a body temperature sensor, a pulse sensor, a respiratory movement sensor, a respiratory flow sensor, a pulse oximetry sensor, a blood pressure meter, a capnograph and/or a maximal oxygen uptake sensor.

According to an embodiment of the invention the one or more sensors 6, 7 comprise one or more activity sensors 7 configured to detect a parameter(s) related to a physical activity of the user. Such parameters include all parameters that describe the duration, intensity and/or type of the activity. Activity sensors 7 allow detecting the physical strain of the user of the audio system 1 in direct as the eventual activity results in a physical strain. Activity sensors 7 and biosensors 6 both may be used exclusively or in combination with each other.

According to an embodiment of the invention the one or more activity sensors 7 comprise a pedometer, a distance meter and/or an activity time keeper.

According to an embodiment of the invention the one or more settings VS, ES, SO, CS1, CS2 comprise a volume control setting VS, an equalizer control setting ES, an audio source selecting setting SO, a dynamic range compression control setting DS and/or an audio content selecting setting CS1, CS2.

The volume control VS setting may be used to set the volume of the audio signal AS at a volume control 13 of the audio player 2. By adapting the volume control setting VS, the volume may be controlled automatically according to the individual perception of the volume of the user under physical stress.

The equalizer control setting ES may be used to control the frequency response of the audio player at an equalizer 14 of the audio player 2. By adapting the equalizer control setting ES, the frequency distribution of the audio signal AS may be controlled automatically according to the individual perception of a frequency distribution of the user under physical stress.

The dynamic range compression control setting DS may be used to set the dynamic range of the audio signal AS at a compressor/expander 16 of the audio player 2. This will affect, amongst others, perceived loudness and clarity. By adapting the dynamic range compression control setting DS, the compression/expansion may be controlled automatically according to the individual perception of the user under physical stress.

An audio source selecting setting SO may be used to select a source signal S1, S2 for further processing in such embodiments wherein the audio player 2 comprises a plurality of audio sources 3 at a source selecting stage 15 of the audio player 2. The select signal S3 may be forwarded to the volume control 13, then forwarded as a volume controlled signal S4 to the equalizer 14 and then forwarded to the compressor/expander 16 as a volume controlled and equalized signal S5. However the order of the volume control 13, the equalizer 14 and/or the compressor/expander 16 may be changed.

Furthermore, an audio content selecting setting CS1, CS2 may be used for controlling the content of the source signal S1, S2 of a certain audio source 3. The term “audio content selecting” refers inter alia to selecting a type of content, e.g. speech or music, to selecting a genre of speech, e.g. news or a fiction, to selecting a genre of music, e.g. beat or soul, to selecting music having specific parameters, e.g. rhythm and speed, or to selecting of a predefined playlist. By adapting the audio content selecting setting CS1, CS2, the audio content of the audio signal AS may be controlled automatically according to the individual perception of audio content of the user under physical stress.

According to an embodiment of the invention the one or more audio sources 3, 4 comprise an audio data receiver 3 configured for providing the source signal S1 based on remote audio data RD. Remote audio data RD are such audio data not stored locally at the audio player 2. The receiver 3 may be an analogous or a digital radio receiver 3 including internet radio receivers or a receiver configured to receive other internet services.

According to an embodiment of the invention the one or more audio sources 3, 4 comprise an audio data reader 4 configured for providing the source signal S2 based on local audio data LD. The audio data reader 4 for example may be an MP3 decoder or a wav file decoder. The audio data may be retrieved from a locally available audio data carrier 11 such as from a hard drive, a solid-state memory, a CD or a DVD.

According to an embodiment of the invention the control device 5 comprises one or more sensors 12 configured for providing the one or more measuring signals MS corresponding to the physical characteristics of ambient sound AB. By these features the audio signal AS may be adapted to the amount and/or characteristics of environmental sound AB.

According to an embodiment of the invention the processor 9 is configured to correlate user preferences UP and values of the measuring signals MS corresponding to the physical characteristics of ambient sound AB, further configured to store the user preferences UP and the correlated values of the physical characteristics of ambient sound AB in the memory 10 and further configured to control the one or more settings VS, ES, SO, CS1, CS2 of the audio player 2 based on the user preferences UP and the correlated values of the physical characteristics of ambient sound AB stored in the memory 10. By these features the audio signal AS may be adapted according to the individual perception of ambient sounds AB by the user.

The object of the invention is further achieved by a method for adaptive audio playback during physical activities comprising the steps of:

providing an audio player 2 having one or more audio sources 3, 4 and being configured to output an audio signal AS based on one or more source signals provided by the one or more audio sources 3, 4; and
providing a control device 5 configured to control one or more settings VS, ES, SO, CS1, CS2 of the of the audio player 2, which affect the audio signal AS;
providing one or more sensor signals SE1, SE2 corresponding to a physical strain of a user of the audio system 1;
inputting user preferences UP for the one or more settings VS, ES, SO, CS1, CS2 of the audio player 2 at a user interface 8 of the control device 5;
correlating user preferences UP and values of the one or more sensor signals SE1, SE2 by using a processor 9 of the control device 5;
storing the user preferences UP and the correlated values of the one or more sensor signals SE1, SE2 in a memory 10 of the control device by using the processor 9 of the control device 5; and
controlling the one or more settings VS, ES, SO, CS1, CS2 of the audio player 2 based on the one or more sensor signals SE1, SE2 and based on the user preferences UP and the correlated values of the one or more sensor signals SE1, SE2 stored in the memory 10 by using the processor 9 of the control device 5.

Moreover, the object of the invention is further achieved by a program for performing, when running on a computer or a processor, the inventive method.

The invention provides an adaptive technical system 1 which automatically readjusts the sound playback based on different relevant factors such as, e.g., degree and type of physical exercise, bio sensory data like maximum oxygen uptake, body temperature, workload or exercise time, individually favored type and amount of counteracting the discomfort, individual hearing preferences. The system 1 may provide for example adaptive loudness and frequency-shaping processing combining bio-sensory data of sports activities with audio signal features. In the same way, an algorithm learning the individual audio-setting preference related to specific physical activities may be provided.

This invention provides a system and method to control the sound output of an electronic system like an audio device, communication device, or multimedia device based on data gathered from bio-sensor and/or direct user feedback which in any case shall be a representation of the physical/perceptual/mental status of the user. The output of the invented system may be a sound signal, which has been processed based on different sets of parameters.

One embodiment of the invention is a system that includes bio-sensors, which measure data related to sport activities and physical exercise (e.g., maximum oxygen uptake, body temperature, heart rate), or sensors detecting the type and workload related to the exercise (jogging distance, duration, etc.). These data are then used by an algorithm which gradually modifies the sound output. For example, the volume or the high-frequency content could be decreased with increasing body temperature and/or with increasing duration of the exercise.

In another embodiment of the invention the system comprises an input interface by means of which the user can enter his or her personally favored settings (e.g., volume control, equalizer settings, playlists, genre, etc.). The system may further comprise a memory to store the settings selected by the user, and an algorithm to relate the settings selected by the user to the input received from sensors such as mentioned above. Over time, the system learns the relation between the selected settings and the sensor data and can automatically adjust the sound processing, thereby acting autonomously without any further action from the user.

In another embodiment of the invention the system comprises at least one microphone, which can pick up sounds from the environment in which the physical exercise takes place. Based on the physical characteristics of the background sound, the system processes the played back audio to keep it audible or to ensure that the user preferences are still met even in the presence of environmental sounds.

Additional embodiments of the invention use combinations of the above mentioned embodiments to produce the processed sound output.

The invented system can significantly improve the comfort of athletes as well as amateurs by reducing their need to readjust the volume during their exercise and by avoiding sound settings that turn uncomfortable during the course of the exercise. As the system may change volume patterns as a result of the automated processing, the frequently described aftereffects of exercising in the presence of audio stimulus (especially loud one), ranging from increased temporal hearing thresholds to permanent threshold shift or/and Tinnitus, could be significantly reduced.

Technical application areas include, but are not limited to:

software and/or hardware for portable audio devices (e.g., mp3-player);
software and/or hardware for stationary audio players used, e.g., for indoor sport activities;
software and/or hardware for portable sound reinforcement systems, e.g., headphones;
software to be run on personal computers to playback audio during physical activities;
special apps for smartphones and other portable devices providing tools and gadgets for sport activities (distance counter, bio-sensors etc.)

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored is on a machine readable carrier.

Other embodiments comprise the computer program for performing one of the methods described herein, stored on a machine readable carrier.

In other words, an embodiment of the inventive method is, therefore, a computer program having a program code for performing one of the methods described herein, when the computer program runs on a computer.

A further embodiment of the inventive method is, therefore, a data carrier (or a digital storage medium, or a computer-readable medium) comprising, recorded thereon, the computer program for performing one of the methods described herein. The data carrier, the digital storage medium or the recorded medium are typically tangible and/or non-transitionary.

A further embodiment of the invention method is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are performed by any hardware apparatus.

While this invention has been described in terms of several advantageous embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1. An audio system for adaptive audio playback during physical activities comprising:
an audio player comprising one or more audio sources and being configured to output an audio signal based on one or more source signals provided by the one or more audio sources; and
a control device configured to control one or more settings of the of the audio player, which affect the audio signal;
wherein the control device comprises a user interface configured to input user preferences for the one or more settings of the audio player;
wherein the control device comprises a processor configured to correlate user preferences and values of one or more sensor signals corresponding to a physical strain of a user of the audio system, further configured to store the user preferences and the correlated values of the one or more sensor signals in a memory of the control device and further configured to control the one or more settings of the audio player based on the one or more sensor signals and based on the user preferences and the correlated values of the one or more sensor signals stored in the memory.

2. An audio system according to claim 1, wherein the one or more sensor signals comprise one or more sensor signals of one or more bio sensors configured to detect a biological parameter of the user.

3. An audio system according to claim 1, wherein the one or more signals of the one or more bio sensors comprise a signal of a body temperature sensor, a signal of a pulse sensor, a signal of a respiratory movement sensor, a signal of a respiratory flow sensor, a signal of a pulse oximetry sensor, a signal of a blood pressure meter, a signal of a capnograph and/or a signal of a maximal oxygen uptake sensor.

4. An audio system according to claim 1, wherein the one or more sensor signals comprise one or more signals of one or more activity sensors configured to detect a parameter related to a physical activity of the user.

5. An audio system according to claim 4, wherein the one or more signals of one or more activity sensors comprise a signal of a pedometer, a signal of a distance meter and/or a signal of an activity time keeper.

6. An audio system according to claim 1, wherein the control device comprises one or more sensors configured to provide the one or more sensor signals corresponding to a physical strain of a user of the audio system.

7. An audio system according to claim 6, wherein the one or more sensors comprise one or more bio a sensors configured to detect a biological parameter of the user.

8. An audio system according to claim 6, wherein the one or more bio sensors comprise a body temperature sensor, a pulse sensor, a respiratory movement sensor, a respiratory flow sensor, a pulse oximetry sensor, a blood pressure meter, a capnograph and/or a maximal oxygen uptake sensor.

9. An audio system according to claim 6, wherein the one or more sensors comprise one or more activity sensors configured to detect a parameter related to a physical activity of the user.

10. An audio system according to claim 6, wherein the one or more activity sensors comprise a pedometer, a distance meter and/or an activity time keeper.

11. An audio system according to claim 1, wherein the one or more settings comprise a volume control setting, an equalizer control setting, an audio source selecting setting, a dynamic range compression control setting and/or an audio content selecting setting.

12. An audio system according to claim 1, wherein the one or more audio sources comprise an audio data receiver configured for providing one of the source signals based on remote audio data.

13. An audio system according to claim 1, wherein the one or more audio is sources comprise an audio data reader configured for providing one of the source signals based on local audio data.

14. An audio system according to claim 1, wherein the processor is configured to control the one or more settings of the audio player based on one or more measuring signals corresponding to physical characteristics of ambient sound.

15. An audio system according to claim 14, wherein the control device comprises one or more sensors configured for providing the one or more measuring signals corresponding to the physical characteristics of ambient sound.

16. An audio system according to claim 15, wherein the processor is configured to correlate user preferences and values of the measuring signals corresponding to the physical characteristics of ambient sound, further configured to store the user preferences and the correlated values of the physical characteristics of ambient sound in the memory and further configured to control the one or more settings of the audio player based on the user preferences and the correlated values of the physical characteristics of ambient sound stored in the memory.

17. A method for adaptive audio playback during physical activities comprising:
providing an audio player comprising one or more audio sources and being configured to output an audio signal based on one or more source signals provided by the one or more audio sources; and
providing a control device configured to control one or more settings of the of the audio player, which affect the audio signal;
providing one or more sensor signals corresponding to a physical strain of a user of the audio system by using one or more sensors of the control device;
inputting user preferences for the one or more settings of the audio player at a user interface of the control device;
correlating user preferences and values of the one or more sensor signals by using a processor of the control device;
storing the user preferences and the correlated values of the one or more sensor signals in a memory of the control device by using the processor of the control device; and
controlling the one or more settings of the audio player based on the one or more sensor signals and based on the user preferences and the correlated values of the one or more sensor signals stored in the memory by using the processor of the control device.

18. A non-transitory digital storage medium having a computer program stored thereon to perform the method for adaptive audio playback during physical activities, the method comprising:
providing an audio player comprising one or more audio sources and being configured to output an audio signal based on one or more source signals provided by the one or more audio sources; and
providing a control device configured to control one or more settings of the of the audio player, which affect the audio signal;
providing one or more sensor signals corresponding to a physical strain of a user of the audio system by using one or more sensors of the control device;
inputting user preferences for the one or more settings of the audio player at a user interface of the control device;
correlating user preferences and values of the one or more sensor signals by using a processor of the control device;
storing the user preferences and the correlated values of the one or more sensor signals in a memory of the control device by using the processor of the control device; and
controlling the one or more settings of the audio player based on the one or more sensor signals and based on the user preferences and the correlated values of the one or more sensor signals stored in the memory by using the processor of the control device,
when said computer program is run by a computer.
